(19) United States
(12) Patent Application Publication
(10) Pub. No.: US 2009/0291058 A1
(43) Pub. Date: Nov. 26, 2009

(54) COSMETIC COMPOSITION COMPRISING AT LEAST ONE ORGANIC SILICON COMPOUND, AT LEAST ONE CATIONIC SURFACTANT AND AT LEAST ONE ORGANIC ACID, AND METHOD FOR COSMETIC TREATMENT OF KERATINOUS FIBERS

(76) Inventors: Frederic WOODLAND, Paris (FR); Pascale LAZZERI, Levallois-Perret (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/430,164

(22) Filed: Apr. 27, 2009

Related U.S. Application Data
(60) Provisional application No. 61/071,655, filed on May 9, 2008.

(30) Foreign Application Priority Data
Apr. 25, 2008 (FR) 0852794

(51) Int. Cl. A61K 8/58; A61Q 5/00

(52) U.S. Cl. 424/70.28; 424/70.27

(57) ABSTRACT

Cosmetic composition and method for the treatment of keratinous fibres. In a cosmetically acceptable medium:
at least one organic silicon compound comprising at least one basic chemical functional group and at least one hydroxyl or hydrolysable group per molecule, said at least one organic silicon compound being chosen from silanes and siloxanes wherein the silanes comprise one silicon atom and are chosen from the compounds of formula (I):

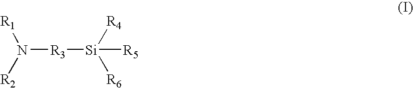

and wherein the siloxanes comprise two or three silicon atoms;
   at least one cationic surfactant; and
   at least one organic acid.

This application claims benefit of U.S. Provisional Application No. 61/071,655, filed May 9, 2008, the contents of which are incorporated herein by reference. This application also claims benefit of priority under 35 U.S.C. § 119 to French Patent Application No. FR 0852794, filed Apr. 25, 2008, the contents of which are also incorporated herein by reference.

The present disclosure relates to a cosmetic composition for the treatment of keratinous fibres, for example, human keratinous fibres, such as the hair, comprising, in a cosmetically acceptable medium, at least one organic silicon compound, at least one cationic surfactant and at least one organic acid. The present disclosure also relates to a method for cosmetic treatment of keratinous fibres.

The hair can be generally damaged and embrittled by the action of external atmospheric agents, such as light and bad weather, and by mechanical or chemical treatments, such as brushing, combing, dyeing, bleaching, perming and/or straightening.

Thus, in an attempt to overcome those disadvantages, it is now common practice to have recourse to hair care products involving the use of care compositions which may make it possible to condition the hair subsequent to these treatments in order to confer thereon, for example, at least one of sheen, softness, suppleness, lightness, a natural feel, and disentangling properties.

These hair care compositions can, for example, be conditioning shampoos or conditioners which can be provided in the form of gels, hair lotions or thick creams.

Furthermore, it has been found that consumers are increasingly looking for care compositions which may make it possible not only to suitably condition the hair but also to provide satisfactory styling effects.

For example, people having fine or curly hair are generally on the lookout for care products that can provide styling effects contributing mass, body and volume to fine hair and defined shape to the curls of wavy hair.

However, conventional care compositions may provide styling effects which can be relatively weak and uneven, for example, in terms of defined curl shape and of volume.

It is known that the introduction of cosmetically active organic compounds, such as cationic polymers and silicones, into care compositions, such as conditioners, may make it possible to confer at least one of disentangling, suppleness, and lightness properties on the hair. However, the styling properties may still remain distinctly inadequate.

Thus there exists a real need to develop cosmetic compositions which may make it possible to satisfactorily condition the hair while contributing powerful styling effects, for example, in terms of at least one of mass, body, volume and defined shape to the curls of the hair.

It has been discovered that it is possible to formulate compositions for the cosmetic treatment of keratinous fibres having at least one of the desired properties, which comprise at least one organic silicon compound as defined below, at least one cationic surfactant and at least one organic acid.

It has been found that the addition of at least one organic silicon compound as disclosed herein to a composition comprising at least one cationic surfactant and at least one organic acid can result in a satisfactory sheathing of the individual hairs, which thus confer a satisfactory soft feel on the hair.

Furthermore, the compositions according to the disclosure may confer powerful styling effects, for example contributing at least one of mass, body and volume to the hair.

Furthermore, the compositions according to the disclosure may make it possible to facilitate the shaping of the hair, for example, of the fine hair.

Finally, the compositions according to the disclosure also may make it possible to confer styling effects on curly hair, for example, in terms of defined shape and control of the curls.

The present disclosure relates, for example, to a cosmetic composition for the treatment of keratinous fibres, for example, human keratinous fibres, such as the hair, comprising, in a cosmetically acceptable medium:

at least one organic silicon compound comprising at least one basic chemical functional group and at least one hydroxyl or hydrolysable group per molecule, said at least one organic silicon compound being chosen from silanes and siloxanes wherein the silanes comprise one silicon atom and are chosen from the compounds of formula (I):

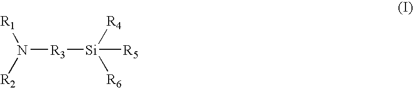

in which:
R₄ represents a halogen or an OR′ or R′₁ group;
R₅ represents a halogen or an OR″ or R′₂ group;
R₆ represents a halogen or an OR′″ or R′₃ group;
R₁, R₂, R₃, R′, R″, R′″, R′₁, R′₂ and R′₃ represent, independently of one another, a saturated or unsaturated, linear or branched hydrocarbon group optionally carrying additional chemical groups;
R₁, R₂, R′, R″ and R′″ can also represent, independently of one another, hydrogen and provided that at least two of the R₄, R₅ and R₆ groups respectively denote OR′, OR″ and OR′″, and at least two of the R′, R″ and R′″ groups are other than hydrogen; and wherein the siloxanes comprise two or three silicone atoms;

at least one cationic surfactant; and

at least one organic acid.

The present disclosure also relates to a method for the cosmetic treatment of keratinous fibres comprising applying to said fibres, the composition according to the disclosure.

It also relates to a method for conditioning keratinous fibres comprising applying to said fibres the composition according to the disclosure.

Other subject-matters and characteristics, aspects and benefits of the disclosure will become apparent on reading the description and examples which follow.

The at least one organic silicon compound used in the composition according to the disclosure is chosen from organosilanes comprising one silicon atom and organosiloxanes comprising two or three silicon atoms, for example, two silicon atoms. They may in addition comprise at least one basic chemical functional group, for example, they may in addition comprise just one basic chemical functional group. The basic chemical functional group can correspond to any functional group which confers a basic nature on the silicon compound, for example, an amine functional group, such as a primary, secondary or tertiary amine functional group. The basic chemical functional group of the silicon compounds according to the disclosure can optionally comprise other functional groups, such as, for example, another amine functional group, an acid functional group or a halogen functional group.

The at least one organic silicon compound used in the composition according to the disclosure may additionally comprise at least one hydrolysable or hydroxyl group per molecule. The at least one hydrolysable group is, for example, alkoxy, aryloxy or halogen group. The at least one silicon compound can also optionally comprise other chemical functional groups, such as acid functional groups.

The at least one organosilane used in the composition according to the disclosure is chosen from the compounds of formula (I):

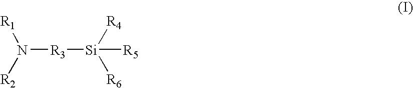

in which:
R₄ represents a halogen or an OR′ or R′₁ group;
R₅ represents a halogen or an OR″ or R′₂ group;
R₆ represents a halogen or an OR′″ or R′₃ group;
R₁, R₂, R₃, R′, R″, R′″, R′₁, R′₂ and R′₃ represent, independently of one another, a saturated or unsaturated, linear or branched hydrocarbon group optionally carrying additional chemical groups,
R₁, R₂, R′, R″ and R′″ can also represent, independently of one another, hydrogen; provided that at least two of the R₄, R₅ and R₆ groups respectively denote OR′, OR″ and OR′″, and at least two of the R′, R″ and R′″ groups are other than hydrogen.

For example, the R₁, R₂, R′, R′₁, R′₂, R′₃, R″ and R′″ groups are chosen from C₁-C₁₂ alkyl, C₆ to C₁₄ aryl, (C₁ to C₈)alkyl(C₆ to C₁₄)aryl and (C₆ to C₁₄)aryl(C₁ to C₈)alkyl radicals.

According to at least one embodiment, the at least one organosiloxane used in the composition according to the disclosure is chosen from the compounds of formula (II):

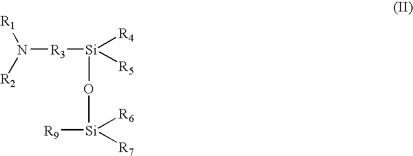

in which:
R₁, R₂, R₃, R₅ and R₆ are defined as above;
R′₄ represents a halogen atom or an OR₁₁ group;
R₇ represents a halogen atom or an OR₁₀ or R″₁ group;
R₉ represents a halogen atom or an OR₈, R″₂ or R₃NR₁R₂ group;
R″₁, R″₂, R₈, R₁₀ and R₁₁ represent a saturated or unsaturated, linear or branched hydrocarbon group optionally carrying additional chemical groups, the R₁₁, R₁₀ and R₈ groups can also represent, independently of one another, a hydrogen atom; provided at least one of the R₆, R₇ and R₉ groups denotes a halogen atom or respectively, an OR′″, OR₁₀ or OR₈ group.

For example, the R″₁, R″₂, R₈, R₁₀ and R₁₁ groups are, independently of one another, chosen from C₁-C₁₂ alkyl, C₆ to C₁₄ aryl, (C₁ to C₈)alkyl(C₆ to C₁₄)aryl and (C₆ to C₁₄)aryl(C₁ to C₈)alkyl radicals.

For example, the halogen atom is a chlorine atom.

The at least one organic silicon compound used in the composition according to the disclosure is, for example, chosen from organosilanes of formula (III):

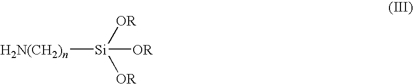

in which the R radicals, which are identical or different, are chosen from C₁-C₆, for example, C₁-C₂, alkyl radicals and n is an integer from 1 to 6, for example, from 2 to 4.

For example, the at least one silane or the siloxane is soluble in water, such as, soluble at a concentration of 2% by weight, further such as at a concentration of 5% by weight and even further such as at a concentration of 10% by weight in water at a temperature of 25° C.±5° C. and at atmospheric pressure. The term “soluble” is understood to mean the formation of a single macroscopic phase.

For example, the at least one organic silicon compound present in the composition according to the disclosure is (3-aminopropyl)triethoxysilane.

The at least one organic silicon compound can be present in the composition according to the disclosure in a content ranging from 0.1 to 20% by weight, such as in a content ranging from 1 to 15% by weight and further such as in a content ranging from 2.5 to 12% by weight, relative to the total weight of the composition.

The composition according to the present disclosure comprises at least one cationic surfactant which are, for example, monomeric.

Mention may be made, as examples of cationic surfactant, of salts of optionally polyoxyalkylenated primary, secondary or tertiary fatty amines and optionally polyoxyalkylenated quaternary ammonium salts.

For example, the at least one cationic surfactant is chosen from optionally polyoxyalkylenated quaternary ammonium salts.

Exemplary mention may be made, as quaternary ammonium salts, of, for example:

those which exhibit the following general formula (IV):

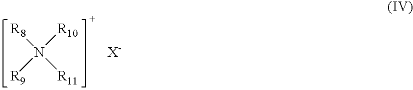

in which the radicals R₈ to R₁₁, which can be identical or different, represent a linear or branched aliphatic radical comprising from 1 to 30 carbon atoms, or an aromatic radical, such as aryl or alkylaryl. The aliphatic radicals can comprise heteroatoms, such as oxygen, nitrogen, sulphur and halogens.

The aliphatic radicals are, for example, chosen from alkyl, alkoxy, polyoxy(C₂-C₆)alkylene, alkylamide, (C₁₂-C₂₂)alkylamido(C₂-C₆)alkyl, (C₁₂-C₂₂)alkyl acetate and hydroxyalkyl radicals comprising approximately from 1 to 30 carbon atoms; X⁻ is an anion chosen from halides, phosphates, acetates, lactates, (C₂-C₆)alkyl sulphates, alkyl- and alkylarylsulphonates;

imidazoline quaternary ammonium salts, such as, for example, those of following formula (V):

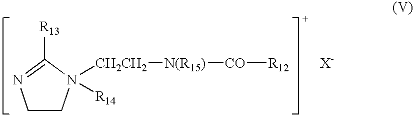

in which R₁₂ represents an alkenyl or alkyl radical comprising from 8 to 30 carbon atoms, for example derivatives of tallow fatty acids, R₁₃ represents a hydrogen atom, a C₁-C₄ alkyl radical or an alkenyl or alkyl radical comprising from 8 to 30 carbon atoms, R₁₄ represents a C₁-C₄ alkyl radical, R₁₅ represents a hydrogen atom or a C₁-C₄ alkyl radical, and X⁻ is an anion chosen from halides, phosphates, acetates, lactates, alkyl sulphates, alkyl- and alkylarylsulphonates. For example, R₁₂ and R₁₃ denote a mixture of alkenyl or alkyl radicals comprising from 12 to 21 carbon atoms, for example derivatives of tallow fatty acids, R₁₄ denotes a methyl radical and R₁₅ denotes a hydrogen atom. Such a product is sold, for example, under the name Rewoquat® W 75 by Rewo;

diquaternary ammonium salts of formula (VI):

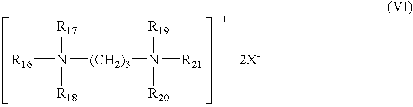

in which R₁₆ denotes an aliphatic radical comprising approximately from 16 to 30 carbon atoms, R₁₇, R₁₈, R₁₉, R₂₀ and R₂₁, which are identical or different, are chosen from hydrogen and an alkyl radical comprising from 1 to 4 carbon atoms, and X⁻ is an anion chosen from halides, acetates, phosphates, nitrates and methyl sulphates. Such diquaternary ammonium salts comprise, for example, propanetallowdiammonium dichloride;

quaternary ammonium salts comprising at least one ester functional group, such as those of following formula (VII):

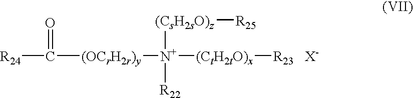

in which:

R₂₂ is chosen from C₁-C₆ alkyl radicals, and C₁-C₆ hydroxyalkyl and dihydroxyalkyl radicals;

R₂₃ is chosen from:

the

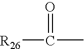

radical

saturated and unsaturated, linear and branched C₁-C₂₂ hydrocarbon radicals R₂₇,

the hydrogen atom,

R₂₅ is chosen from:

the

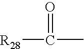

radical

saturated and unsaturated, linear and branched C₁-C₆ hydrocarbon radicals R₂₉,

the hydrogen atom,

R₂₄, R₂₆ and R₂₈, which are identical or different, are chosen from saturated and unsaturated, linear and branched C₇-C₂₁ hydrocarbon radicals;

r, s and t, which are identical or different, are integers ranging from 2 to 6;

y is an integer having a value from 1 to 10;

x and z, which are identical or different, are integers having values from 0 to 10;

X⁻ is an organic or inorganic, simple or complex anion;

with the proviso that the sum x+y+z has a value from 1 to 15, that, when x has a value of 0, then R₂₃ denotes R₂₇ and that, when z has a value of 0, then R₂₅ denotes R₂₉.

The R₂₂ alkyl radicals can be linear or branched, such as linear.

For example, R₂₂ denotes a methyl, ethyl, hydroxyethyl or dihydroxypropyl radical, such as, a methyl or ethyl radical.

For example, the sum x+y+z has a value from 1 to 10.

When R₂₃ is an R₂₇ hydrocarbon radical, it can be long and have from 12 to 22 carbon atoms, or short and have from 1 to 3 carbon atoms.

When R₂₅ is an R₂₉ hydrocarbon radical, it, for example, has from 1 to 3 carbon atoms.

For example, R₂₄, R₂₆ and R₂₈, which are identical or different, are chosen from saturated and unsaturated, linear and branched C₁₁-C₂₁ hydrocarbon radicals, such as from saturated and unsaturated, linear and branched C₁₁-C₂₁ alkyl and alkenyl radicals.

For example, x and z, which are identical or different, have values of 0 or 1.

For example, y is equal to 1.

For example, r, s and t, which are identical or different, have values of 2 or 3, for example, a value of 2.

The anion is, for example, a halide (chloride, bromide or iodide) or an alkyl sulphate, such as methyl sulphate. However, non-limiting mention may be made of methanesulphonate, phosphate, nitrate, tosylate, an anion derived from an organic acid, such as acetate or lactate, and any other anion compatible with the ammonium comprising an ester functional group.

The anion X⁻ is, for example, chloride or methyl sulphate.

Exemplary use may be made, in the composition according to the disclosure, of the ammonium salts of formula (VII) in which:

R₂₂ denotes a methyl or ethyl radical,

x and y are equal to 1;

z is equal to 0 or 1;

r, s and t are equal to 2;

R₂₃ is chosen from:

the

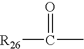

radical

methyl, ethyl or C₁₄-C₂₂ hydrocarbon radicals, and

the hydrogen atom;

R₂₃ is chosen from:

the

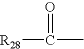

radical, and

the hydrogen atom;

R₂₄, R₂₆ and R₂₈, which are identical or different, are chosen from saturated and unsaturated, linear and branched C₁₃-C₁₇ hydrocarbon radicals, such as from saturated and unsaturated, linear and branched C₁₃-C₁₇ alkyl and alkenyl radicals.

For example, the hydrocarbon radicals are linear.

Non-limiting mention may be made, for example, of compounds of formula (VII), such as diacyloxyethyldimethylammonium, diacyloxyethyl-(hydroxyethyl)methylammonium, monoacyloxyethyldi(hydroxyethyl)methylammonium, triacyloxyethylmethylammonium and monoacyloxyethyl(hydroxyethyl)dimethylammonium salts (such as chloride or methyl sulphate), and their mixtures. The acyl radicals, for example, have from 14 to 18 carbon atoms and originate, for example, from a vegetable oil, such as palm oil or sunflower oil. When the compound comprises several acyl radicals, the latter can be identical or different.

These products are obtained, for example, by direct esterification of triethanolamine, triisopropanolamine, alkyldiethanolamine or alkyldiisopropanolamine, optionally oxyalkylenated, with fatty acids or with mixtures of fatty acids of vegetable or animal origin, or by transesterification of their methyl esters. This esterification is followed by quaternization using an alkylating agent, such as an alkyl halide (such as methyl or ethyl halide), a dialkyl sulphate (such as dimethyl or diethyl sulphate), methyl methane-sulphonate, methyl para-toluenesulphonate, glycol chlorohydrin or glycerol chlorohydrin.

Such compounds are sold, for example, under the names Dehyquart® by Henkel, Stepanquat® by Stepan, Noxamium® by Ceca or Rewoquat® WE 18 by Rewo-Witco.

The composition according to the disclosure, for example, comprises a mixture of quaternary ammonium mono-, di- and triester salts with a predominance by weight of diester salts.

Non-limiting use may be made, as mixture of ammonium salts, for example, of the mixture comprising from 15 to 30% by weight of acyloxyethyl-di(hydroxyethyl)methylammonium methyl sulphate, from 45 to 60% by weight of diacyloxyethyl(hydroxyethyl)methylammonium methyl sulphate and from 15 to 30% by weight of triacyloxyethylmethylammonium methyl sulphate, the acyl radicals having from 14 to 18 carbon atoms and originating from palm oil which is optionally partially hydrogenated.

Exemplary use may also be made of the ammonium salts comprising at least one ester functional group described in U.S. Pat. Nos. 4,874,554 and 4,137,180.

Non-limiting mention may be made, among quaternary ammonium salts of formula (IV), on the one hand, of tetraalkylammonium chlorides, such as, for example, dialkyldimethylammonium or alkyltrimethylammonium or alkylaralkyldimethylammonium chlorides in which the alkyl radical comprises approximately from 12 to 22 carbon atoms, for example, behenyltrimethylammonium chloride, distearyldimethylammonium chloride, cetyltrimethylammonium chloride or benzyldimethylstearylammonium chloride, or also, on the other hand, of palmitamidopropyltrimethylammonium chloride or stearamidopropyl-dimethyl(myristyl acetate)ammonium chloride, sold under the name Ceraphyl® 70 by Van Dyk.

For example, the at least one cationic surfactant used in the composition according to the disclosure is chosen from behenyltrimethylammonium and cetyltrimethylammonium chlorides and their mixtures.

The at least one cationic surfactant used in the composition according to the present disclosure can be present in the composition in an amount varying from 0.1 to 6% by weight, such as in an amount varying from 0.5 to 3% by weight.

The term “organic acid” is understood to mean any nonpolymeric organic compound comprising at least one acid functional group chosen from carboxylic acid, sulphonic acid and phosphoric acid functional groups.

For example, the organic acid is not a surfactant.

For example, the molecular weight of the organic acid is less than 250, such as less than 200.

The organic acids can be amino acids.

The at least one organic acid is, for example, chosen from acetic acid, propanoic acid, butanoic acid, lactic acid, malic acid, glycolic acid, ascorbic acid, maleic acid, phthalic acid, succinic acid, taurine, tartaric acid, arginine, glycine, glucuronic acid, gluconic acid and citric acid.

for further example, the at least one organic acid according to the disclosure iscarboxylic acid.

for even further example, the at least one organic acid used in the composition according to the disclosure is chosen form acetic acid, citric acid and lactic acid.

In the composition, the at least one organic acid can be in the free or salified form.

The at least one organic acid used in the composition according to the present disclosure can be present in a content, expressed as free acids, ranging from 0.1 to 10% by weight, such as in a content ranging from 0.5 to 8% by weight and further such as in a content ranging from 1 to 5% by weight, with respect to the total weight of the composition.

The composition according to the present disclosure can also comprise at least one additional surfactant chosen from anionic, amphoteric and nonionic surfactants.

The at least one anionic surfactant which can be used in the compositions of the disclosure is chosen, for example, from the salts, such as alkali metal salts, such as sodium salts, ammonium salts, amine salts, aminoalcohol salts and alkaline earth metal salts, for example magnesium salts, of the following types: alkyl sulphates, alkyl ether sulphates, alkylamido ether sulphates, alkylaryl polyether sulphates, monoglyceride sulphates, alkylsulphonates, alkylamidesulphonates, alkylarylsulphonates, □-olefinsulphonates, paraffinsulphonates, alkyl sulphosuccinates, alkyl ether sulphosuccinates, alkylamide sulphosuccinates, alkyl sulphoacetates, acylsarcosinates and acylglutamates, the alkyl and acyl groups of all these compounds comprising from 6 to 24 carbon atoms and the aryl group, for example, denoting a phenyl or benzyl group.

Exemplary use may also be made of C₆₋₂₄ alkyl monoesters of polyglycosidedicarboxylic acids, such as alkyl glucosidecitrates, alkyl polyglycosidetartrates and alkyl polyglycosidesulphosuccinates, alkyl sulphosuccinamates, acylisethionates and N-acyltaurates, the alkyl or acyl group of all these compounds comprising from 12 to 20 carbon atoms.

Another group of anionic surface-active agents which can used in the compositions of the present disclosure is that of the acyl lactylates, the acyl group of which comprises from 8 to 20 carbon atoms.

In addition, exemplary mention may also be made of alkyl-D-galactoside-uronic acids and their salts, and also polyoxyalkylenated C₆₋₂₄ alkyl ether carboxylic acids, polyoxyalkylenated (C₆₋₂₄)alkyl(C₆₋₂₄)aryl ether carboxylic acids, polyoxyalkylenated (C₆₋₂₄)alkylamido ether carboxylic acids and their salts, such as those comprising from 2 to 50 ethylene oxide units, and their mixtures.

Exemplary use is made of alkyl sulphates, alkyl ether sulphates and their mixtures, for example, in the form of alkali metal, alkaline earth metal, ammonium, amine or aminoalcohol salts.

When they are present, the amount of the at least one anionic surfactant is for example, within the range from 0.1 to 4% by weight, relative to the total weight of the composition.

For example, the composition does not comprise anionic surfactants.

Non-limiting examples of additional nonionic surfactants which can be used in the composition of the present disclosure are described for example in “Handbook of Surfactants” by M. R. Porter, published by Blackie & Son (Glasgow and London), 1991, pp. 116-178. They are chosen, for example, from polyethoxylated, polypropoxylated and polyglycerolated fatty alcohols, alpha-diols, (C₁₋₂₀)alkylphenols and acids having a fatty chain comprising, for example, from 8 to 18 carbon atoms, it being possible for the number of ethylene oxide or propylene oxide groups to range, for example, from 2 to 50 and it being possible for the number of glycerol groups to range, for example, from 2 to 30.

Non-limiting mention may also be made of condensates of ethylene oxide and of propylene oxide with fatty alcohols; polyethoxylated fatty amides, for example, having from 2 to 30 ethylene oxide units, polyglycerolated fatty amides comprising on average from 1 to 5 glycerol groups and, for example, from 1.5 to 4, ethoxylated sorbitan fatty acid esters having from 2 to 30 ethylene oxide units, sucrose fatty acid esters, polyethylene glycol fatty acid esters, (C₆₋₂₄ alkyl)polyglycosides, N—(C₆₋₂₄ alkyl)glucamine derivatives or amine oxides, such as (C₁₀₋₁₄ alkyl)amine oxides or N—(C₁₀₋₁₄ acyl)amino-propylmorpholine oxides.

When it is present, the amount of the additional at least one nonionic surfactant is, for example, within the range from 0.01 to 20% by weight, such as from 0.1 to 10% by weight, with respect to the total weight of the composition.

The amphoteric or zwitterionic surface-active agents which can be used in the present disclosure can, for example, be aliphatic secondary or tertiary amine derivatives in which the aliphatic group is a linear or branched chain comprising from 8 to 22 carbon atoms and comprising at least one anionic group, such as, for example, a carboxylate, sulphonate, sulphate, phosphate or phosphonate group. Exemplary mention may be made of (C₈₋₂₀)alkyl betaines, sulphobetaines, (C₈₋₂₀ alkyl) amido(C₆₋₈ alkyl) betaines or (C₈₋₂₀ alkyl) amido(C₆₋₈ alkyl) sulphobetaines.

Exemplary mention may be made, among amine derivatives, of the products sold under the name Miranol®, such as described in U.S. Pat. Nos. 2,528,378 and 2,781,354 and classified in the CTFA dictionary, 3rd Edition, 1982, under the names Amphocarboxyglycinate and Amphocarboxypropionate with the respective structures (A) and (B):

Rₐ—CONHCH₂CH₂—N(R_{b})(R_{c})(CH₂COO⁻) (A)

in which:

Rₐ represents an alkyl group derived from an acid Rₐ—COOH present in hydrolysed coconut oil or a heptyl, nonyl or undecyl group,

R_{b} represents a β-hydroxyethyl group, and

R_{c} represents a carboxymethyl group;

and

Rₐ′—CONHCH₂CH₂—N(B)(B′) (B)

in which:

B represents —CH₂CH₂OX′,

B′ represents —(CH₂)_{z}—Y′, with z=1 or 2,

X′ represents the —CH₂CH₂—COOH group or a hydrogen atom,

Y′ represents —COOH or the —CH₂—CHOH—SO₃H group,

Rₐ′ represents an alkyl group of an acid Rₐ′—COOH present in hydrolysed coconut oil or in hydrolysed linseed oil, an alkyl group, such as a C₁₇ alkyl group and its iso form, or an unsaturated C₁₇ group.

These compounds are classified in the CTFA dictionary, 5th Edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

Mention may be made, by way of non-limiting example, of the cocoamphodiacetate sold by Rhodia under the trade name Miranol® C2M Concentrate.

Exemplary use is made, among the amphoteric or zwitterionic surfactants mentioned above, of (C₈₋₂₀)alkyl betaines, (C₈₋₂₀ alkyl) amido(C₆₋₈ alkyl) betaines and their mixtures.

When they are present, the amount of the amphoteric or zwitterionic surfactant or surfactants is, for example, within the range from 0.01 to 20% by weight, such as from 0.1 to 10% by weight, with respect to the total weight of the composition.

The term “cosmetically acceptable medium” is understood to mean a medium compatible with keratinous fibres, such as the hair.

The cosmetically acceptable medium is composed of water or of a mixture of water and of at least one cosmetically acceptable solvent chosen from lower C₁-C₄ alcohols, such as ethanol, isopropanol, tert-butanol and n-butanol; polyols, such as glycerol, propylene glycol and polyethylene glycol.

The pH of the compositions according to the disclosure can generally varie from 3 to 11, such as from 7 to 10.

The composition according to the disclosure can additionally comprise at least one conventional additive well known in the art, such as: natural or synthetic thickeners or viscosity regulators; C₁₂-C₃₀ fatty alcohols; ceramides; oily fatty esters, such as isopropyl myristate, myristyl myristate, cetyl palmitate and stearyl stearate; mineral, vegetable or synthetic oils, such as alpha-olefins or palm oil; vitamins or provitamins; cationic or amphoteric polymers; pH-stabilizing agents; preservatives; and colorants.

The at least one thickening agent can be chosen from cellulose thickening agents, for example hydroxyethylcellulose, hydroxypropylcellulose and carboxymethylcellulose, guar gum and its derivatives, for example hydroxypropyl guar, sold by Rhodia under the reference Jaguar HP 105, gums of microbial origin, such as xanthan gum and scleroglucan gum, synthetic thickening agents, such as crosslinked homopolymers of acrylic acid or of acrylamidopropanesulphonic acid, for example Carbomer, or nonionic, anionic, cationic or amphoteric associative polymers, such as the polymers sold under the names Pemulen TR1 or TR2 by Goodrich, Salcare SC90 by Ciba, Aculyn 22, 28, 33, 44 or 46 by Röhm & Haas and Elfacos T210 and T212 by Akzo.

For example, the at least one additive is chosen from cationic polymers and thickening agents, further for example from non-ionic associative polymers, such as the polyurethanes, for example the polyurethanes sold under the name Aculyn 44 and Aculyn 46.

A person skilled in the art will take care to choose the optional additives and their amounts so that they do not harm the properties of the compositions of the present disclosure.

The at least one additive can be generally present in the composition according to the disclosure in an amount ranging from 0 to 20% by weight, with respect to the total weight of the composition.

The compositions in accordance with the disclosure can be used for the conditioning of keratinous fibres, such as the hair, for example as conditioners.

For example, the compositions of the invention are rinse-out or leave-in conditioners.

Another aspect of the disclosure is a method for the cosmetic treatment of keratinous fibres, such as the hair, which comprises applying an effective amount of a composition as described above to the said fibres, and optionally rinsing after an optional leave-in time.

The following examples are intended to illustrate the present disclosure without however limiting the scope thereof.

### EXAMPLE 1

Compositions (A), (B), (C) and (D) were prepared from the ingredients shown in the table below, the amounts of which were expressed as percent by weight of active materials, with respect to the total weight of the composition.

| Compositions | A (invention) | B (invention) | C (invention) | D (comparative) |
|---|---|---|---|---|
| 3-Aminopropyltriethoxysilane | 10 | 10 | 10 | — |
| Cetearyl alcohol (C₁₆/C₁₈ 50/50) | 2.5 | 3 | 5 | 5 |
| Mixture of myristyl myristate, | 0.5 | — | — | — |
| cetyl palmitate and stearyl | | | | |
| stearate | | | | |
| Palm oil | — | 2 | — | — |
| Behenyltrimethyl ammonium | 1.2 | — | 2.4 | 2.4 |
| chloride | | | | |
| Cetyltrimethylammonium | — | 0.8 | — | — |
| chloride | | | | |
| Lactic acid | 4 | 4 | 0.5 | 0.5 |
| Water | q.s. for | q.s. for | q.s. for | q.s. for |
| | 100% | 100% | 100% | 100% |

Applied as conditioners, compositions (A) and (B) confered mass, volume and a satisfactory soft feel on the hair.

Furthermore, these compositions made it possible to facilitate the shaping of fine hair and to contribute a better curl definition to curly hair.

A comparative trial between compositions (C) and (D) showed that composition (C) contributed more body to the hair than composition (D).

### EXAMPLE 2

Compositions (E), (F), (G) and (H) were prepared from the ingredients shown in the table below, the amounts of which were expressed as percent by weight of active materials, with respect to the total weight of the composition.

| Compositions | E | F | G | H |
|---|---|---|---|---|
| 3-Aminopropyltriethoxysilane | 10 | 10 | 2 | 2 |
| Cetearyl alcohol (C₁₆/C₁₈ 50/50) | 4 | 4.75 | 4 | 4.75 |
| Mixture of myristyl myristate, cetyl | | 1 | | 1 |
| palmitate and stearyl stearate | | | | |
| PEG-150/STEARYL | 0.3 | 0.375 | 0.3 | 0.375 |
| ALCOHOL/SMDI COPOLYMER | | | | |
| (ACULYN 46 from | | | | |
| ROHM&HAAS) | | | | |
| POLYQUATERNIUM-37 | 0.5 | 0.5 | 0.5 | 0.5 |
| (SALCARE SC 95 from CIBA) | | | | |
| Behentrimonium Chloride | 0.4 | | 0.4 | |
| (GENAMIN KDMP from | | | | |
| CLARIANT) | | | | |
| Cetrimonium Chloride | | 0.25 | | 0.25 |
| (DEHYQUART A OR from | | | | |
| COGNIS) | | | | |
| Lactic acid | 3.5 | 3.5 | 0.7 | 0.7 |
| | pH 9 ± 0.3 | pH 9 ± 0.3 | pH 9 ± 0.3 | pH 9 ± 0.3 |
| Water | Qsp 100 | Qsp 100 | Qsp 100 | Qsp 100 |

Applied as conditioners, compositions (E), (F), (G) and (H) conferred mass, volume and a satisfactory soft feel on the hair.

Furthermore, these compositions made it possible to facilitate the shaping of fine hair and to contribute a better curl definition to curly hair.

What is claimed is:

1. A cosmetic composition for the treatment of keratinous fibres, comprising, in a cosmetically acceptable medium:
at least one organic silicon compound comprising at least one basic chemical functional group and at least one hydroxyl or hydrolysable group per molecule, said at least one organic silicon compound being chosen from silanes and siloxanes,
wherein the silanes comprise one silicon atom and are chosen from the compounds of formula (I): 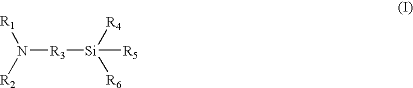
in which:
R₄ represents a halogen or an OR′ or R′₁ group;
R₅ represents a halogen or an OR″ or R′₂ group;
R₆ represents a halogen or an OR′″ or R′₃ group;
R₁, R₂, R₃, R′, R″, R′″, R′₁, R′₂ and R′₃ represent, independently of one another, a saturated or unsaturated, linear or branched hydrocarbon group optionally carrying at least one additional chemical group;
R₁, R₂, R′, R″ and R′″ can also represent, independently of one another, hydrogen;
provided that at least two of the R₄, R₅ and R₆ groups respectively denote OR′, OR″ and OR′″, and at least two of the R′, R″ and R′″ groups are other than hydrogen;
and wherein the siloxanes comprise two or three silicone atoms;
at least one cationic surfactant; and
at least one organic acid.

2. The cosmetic composition according to claim 1, wherein the at least one basic chemical functional group of the at least one organic silicon compound is independently chosen from primary, secondary and tertiary amines.

3. The cosmetic composition according to claim 1, wherein the at least one hydrolysable group of the at least one organic silicon compound is independently chosen from alkoxy, aryloxy and halogen groups.

4. The cosmetic composition according to claim 1, wherein the siloxanes are chosen from the compounds of formula (II): 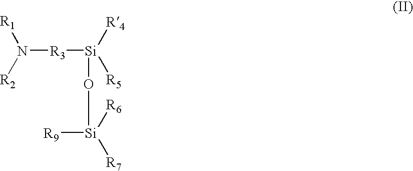
in which:
R₅ represents a halogen or an OR″ or R′₂ group;
R₆ represents a halogen or an OR′″ or R′₃ group;
R₁, R₂, R₃, R″, R′″, R′₂ and R′₃ represent, independently of one another, a saturated or unsaturated, linear or branched hydrocarbon group optionally carrying additional chemical groups,
R₁, R₂, R″ and R′″ can also represent, independently of one another, hydrogen;
R′₄ represents a halogen atom or an OR₁₁ group;
R₇ represents a halogen atom or an OR₁₀ or R″₁ group;
R₉ represents a halogen atom or an OR₈, R″₂ or R₃NR₁R₂ group;
R″₁, R″₂, R₈, R₁₀ and R₁₁ represent a saturated or unsaturated, linear or branched hydrocarbon group optionally carrying additional chemical groups,
the R₁₁, R₁₀ and R₈ groups can also represent, independently of one another, a hydrogen atom;
provided that at least one of the R₆, R₇ and R₉ groups denote a halogen atom or, respectively, an OR′″, OR₁₀ or OR₈ group.

5. The cosmetic composition according to claim 4, wherein the R₁, R₂, R′, R′₁, R′₂, R′₃, R″, R′″, R″₁, R″₂, R₈, R₁₀ and R₁₁ groups are, independently of one another, chosen from C₁-C₁₂ alkyl, C₆ to C₁₄ aryl, (C₁ to C₈)alkyl(C₆ to C₁₄)aryl and (C₆ to C₁₄)aryl(C₁ to C₈)alkyl radicals.

6. The cosmetic composition according to claim 1, wherein the silanes are chosen from the compounds of formula (III): 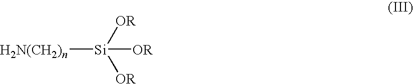
in which the R radicals, which are identical or different, are chosen from C₁-C₆ alkyl radicals and n is an integer ranging from 1 to 6.

7. The cosmetic composition according to claim 6, wherein the n is an integer ranging from 2 to 4.

8. The cosmetic composition according to claim 1, wherein the at least one organic silicon compound is (3-aminopropyl)triethoxysilane.

9. The cosmetic composition according to claim 1, wherein the at least one organic silicon compound is present in an amount ranging from 0.1 to 20% by weight relative to the total weight of the composition.

10. The cosmetic composition according to claim 9, wherein the at least one organic silicon compound is present in an amount ranging from 2.5 to 12% by weight relative to the total weight of the composition.

11. The cosmetic composition according to claim 1, wherein the at least one cationic surfactant is chosen from salts of optionally polyoxyalkylenated primary, secondary and tertiary fatty amines, and optionally polyoxyalkylenated quaternary ammonium salts.

12. The cosmetic composition according to claim 11, wherein the at least one quaternary ammonium salt is chosen from:
compounds of formula (IV): 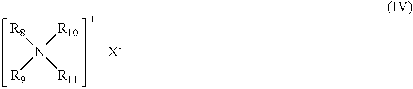
in which the radicals R₈ to R₁₁, being identical or different, represent a linear or branched aliphatic radical comprising from 1 to 30 carbon atoms, or an aromatic radical; and X⁻ is an anion chosen from halides, phosphates, acetates, lactates, (C₂-C₆)alkyl sulphates, alkyl- and alkylarylsulphonates;
imidazoline quaternary ammonium salts;
diquaternary ammonium salts of formula (VI): 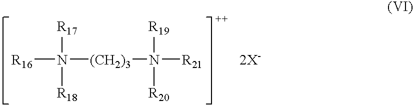
in which R₁₆ represents an aliphatic radical comprising from 16 to 30 carbon atoms, R₁₇, R₁₈, R₁₉, R₂₀ and R₂₁, being identical or different, are chosen from hydrogen and C₁-C₄ alkyl radicals, and X⁻ is an anion chosen from halides, acetates, phosphates, nitrates and methyl sulphates; and
quaternary ammonium salts comprising at least one ester functional group.

13. The cosmetic composition according to claim 1, wherein the at least one cationic surfactant is present in an amount ranging from 0.1 to 6% by weight relative to the total weight of the composition.

14. The cosmetic composition according to claim 14, wherein the at least one cationic surfactant is present in an amount ranging from 0.5 to 3% by weight relative to the total weight of the composition.

15. The cosmetic composition according to claim 1, wherein the at least one organic acid is chosen from acetic acid, propanoic acid, butanoic acid, lactic acid, glycolic acid, ascorbic acid, maleic acid, phthalic acid, succinic acid, taurine, tartaric acid, arginine, glycine, gluconic acid, glucuronic acid and citric acid.

16. The cosmetic composition according to claim 1, wherein the at least one organic acid is present in an amount ranging from 0.1 to 10% by weight relative to the total weight of the composition.

17. The cosmetic composition according to claim 17, wherein the at least one organic acid is present in an amount ranging from 1 to 5% by weight relative to the total weight of the composition.

18. A method for the cosmetic treatment of keratinous fibres, comprising:
applying to the keratinous fibres a cosmetic composition; and
rinsing the keratinous fibres after an optional leave-in time of the cosmetic composition;
wherein the cosmetic composition comprises, in a cosmetically acceptable medium:
at least one organic silicon compound comprising at least one basic chemical functional group and at least one hydroxyl or hydrolysable group per molecule, said at least one organic silicon compound being chosen from silanes and siloxanes,
wherein the silanes comprise one silicon atom and are chosen from the compounds of formula (I): 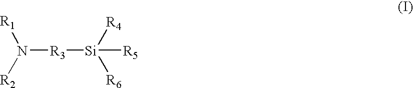
in which:
R₄ represents a halogen or an OR′ or R′₁ group;
R₅ represents a halogen or an OR″ or R′₂ group;
R₆ represents a halogen or an OR′″ or R′₃ group;
R₁, R₂, R₃, R′, R″, R′″, R′₁, R′₂ and R′₃ represent, independently of one another, a saturated or unsaturated, linear or branched hydrocarbon group optionally carrying at least one additional chemical group;
R₁, R₂, R′, R″ and R′″ can also represent, independently of one another, hydrogen;
provided that at least two of the R₄, R₅ and R₆ groups respectively denote OR′, OR″ and OR′″, and at least two of the R′, R″ and R′″ groups are other than hydrogen;
and wherein the siloxanes comprise two or three silicone atoms;
at least one cationic surfactant; and
at least one organic acid.

19. A method for conditioning keratinous fibres, comprising:
applying to the keratinous fibres a cosmetic composition,
wherein the cosmetic composition comprises, in a cosmetically acceptable medium:
at least one organic silicon compound comprising at least one basic chemical functional group and at least one hydroxyl or hydrolysable group per molecule, said at least one organic silicon compound being chosen from silanes and siloxanes,
wherein the silanes comprise one silicon atom and are chosen from the compounds of formula (I): 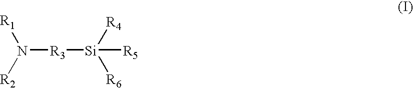
in which:
R₄ represents a halogen or an OR′ or R′₁ group;
R₅ represents a halogen or an OR″ or R′₂ group;
R₆ represents a halogen or an OR′″ or R′₃ group;
R₁, R₂, R₃, R′, R″, R′″, R′₁, R′₂ and R′₃ represent, independently of one another, a saturated or unsaturated, linear or branched hydrocarbon group optionally carrying at least one additional chemical group;
R₁, R₂, R′, R″ and R′″ can also represent, independently of one another, hydrogen;
provided that at least two of the R₄, R₅ and R₆ groups respectively denote OR′, OR″ and OR′″, and at least two of the R′, R″ and R′″ groups are other than hydrogen;
and wherein the siloxanes comprise two or three silicone atoms;
at least one cationic surfactant; and
at least one organic acid.
